(19) United States
(12) Patent Application Publication
(10) Pub. No.: US 2016/0030720 A1
(43) Pub. Date: Feb. 4, 2016

(54) NOSE BLEED TREATMENT SYSTEM

(71) Applicant: Abbas M. Husain, Voorhees, NJ (US)

(72) Inventor: Abbas M. Husain, Voorhees, NJ (US)

(21) Appl. No.: 14/463,624

(22) Filed: Aug. 19, 2014

Related U.S. Application Data
(63) Continuation-in-part of application No. 14/445,526, filed on Jul. 29, 2014.

(51) Int. Cl. A61M 31/00; A61K 38/36; A61K 31/137; A61B 17/08; A61K 31/122

(52) U.S. Cl. CPC A61M 31/00; A61B 17/083; A61K 31/122; A61K 31/137; A61K 38/363

(57) ABSTRACT

A nose bleed treatment system for use in treating a patient having a nose bleed uses a nose clip that has a swab adjustment holder disposed on it to moveably hold a bulky or oversized swab within it. The swab can be moved to press against a bleed site in a patient having a bloody nose. A medically effective agent is applied to the bleed site either by applying it to the swap or by using a specially designed spray bottle. In the embodiment using the spray bottle, the bottle has an elongated tip that allows the user to position the tip of the spray bottle proximal to the bleed site. The medically effective agent contains at least one of a clot enhancing agent and a vasoconstricting agent. The agent may contain vitamin K as well as a vasoconstricting agent epinephrine.

This is a continuation in part of U.S. patent application Ser. No. 14/445,526, filed Jul. 29, 2014, the complete disclosure of which is hereby incorporated by reference.

### BACKGROUND OF THE INVENTION

In the United States, many people develop a nose bleed at some time in their lives. Some statistics put it at one out of seven people will experience a nose bleed. Most nose bleeds involve anterior blood vessels and can be safely treated. Nose bleeds that involve posterior blood vessels are more serious and generally require professional treatment as they are generally difficult to reach.

There is a need for a safe, easy to use nose bleed treatment system that treats a nose bleed using both mechanical and biological means and allows the user to reach the affected portion of the nose.

### SUMMARY OF THE INVENTION

A nose bleed treatment system for use in treating a patient having a nose bleed uses a nose clip that has a swab adjustment holder disposed on it to moveably hold a bulky or oversized swab within it. The swab can be moved to press against a bleed site in a patient having a bloody nose. A medically effective agent is applied to the bleed site either by applying it to the swap or by using a specially designed spray bottle. In the embodiment using the spray bottle, the bottle has an elongated tip that allows the user to position the tip of the spray bottle proximal to the bleed site. The medically effective agent contains at least one of a clot enhancing agent and a vasoconstricting agent. The agent may contain vitamin K as well as a vasoconstricting agent epinephrine.

Other features and advantages of the instant invention will become apparent from the following description of the invention which refers to the accompanying drawings.

### BRIEF DESCRIPTION OF THE DRAWINGS

FIG. 1 is an illustration of a nose bleed treatment system clip according to an embodiment of the invention.

FIG. 2 is an illustration of a nose bleed treatment system clip according to an embodiment of the invention.

FIG. 3 is an illustration of a packaging means for the nose bleed treatment system shown in FIG. 2.

FIG. 4 is an illustration of the nose bleed treatment system shown in FIG. 1 with a sealed swab.

FIG. 5 is an illustration of the nose bleed treatment system shown in FIG. 1 with a medication packet.

FIG. 6 is an illustration of the nose bleed treatment system shown in FIG. 1 with medication applied to swab.

FIG. 7 is an illustration of a nose bleed treatment system according to an embodiment of the invention.

FIG. 8 is an illustration of nose spray bottles according embodiments of the invention.

FIG. 9 is an illustration of a user applying spray to a posterior portion of a nose bleed.

FIG. 10 is an illustration of a user applying spray to an anterior portion of a nose bleed.

FIG. 11 is a top view of a tip of a nose spray bottle according to an embodiment of the invention.

FIG. 12 is an illustration of a nose clip according to an embodiment of the present invention.

FIG. 13 is an illustration of a nose clip according to an embodiment of the present invention having two swabs.

FIG. 14 is an illustration of the nose clip shown in FIG. 12 in use with a patient.

FIG. 15 is a front hidden view showing the nose clip shown in FIG. 12 positioned within nasal cavity.

FIG. 16 is illustration of a nose bleed treatment system clip according to an embodiment of the invention having a bulky cotton swab.

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

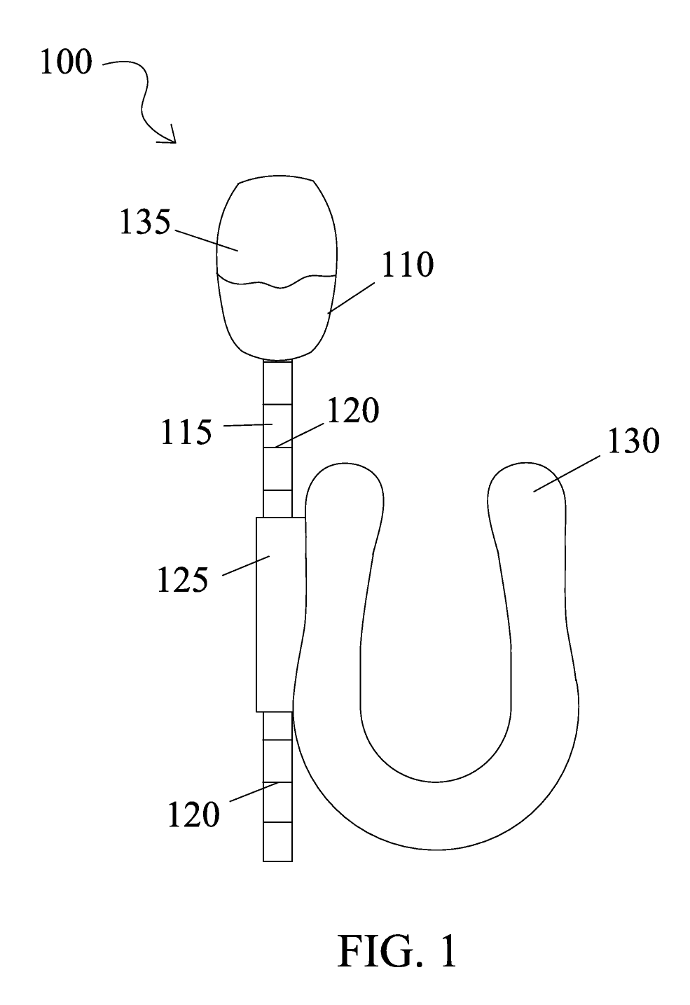

Referring to FIG. 1, a nose bleed treatment system 100 has a nose clip 130 with a swab holder portion 125 that allows a user to move a swab 115 into the desired position in a user's nostril that is bleeding. Swab 115 may be moved up and down within swab holder 125. Swab 115 has a tip portion 110 that is made of an absorbent material that soaks up and contains the blood (not shown). To facilitate stopping the nose bleed, a medication 135 is applied to swab tip 110. Clip 130 is resiliently held in place by stretching the clip over the nostrils and then releasing which clamps the nostrils together to help stop the bleeding.

Medication 135 is preferably a clot enhancing agent containing vitamin K as well as a vasoconstricting agent. Medication 135 may be, but not limited to, epinephrine with pytonadione (vitamin K1), menaquinone (vitamin K2), menadion (vitamin K3) fibrogen or other suitable medications. The medication acts biologically to stop the bleed and clip 130 provides for mechanical pressure to stop the flow of blood. The ingredients are preferably all natural and may be homeopathic in scope. The medication should contain at least one effective dose of the above ingredients and may contain a combination.

It is important to note that there are very effective herbal sources for the medication used in the spray that contain natural ephedra compounds which act as vasoconstricting agents like phenylephrine/ephedrine. Also, herbal sources containing vitamin K and K2 compounds are useful and natural. Additionally, mammalian sources such as cattle or pig may be used as a source of serums containing fibrinogen that facilitates and enhances blood clotting.

If the blood flow is excessive, a new swab 115 may be inserted taking the place of the saturated one. A series of graduated lines 120 are provided to allow the user to determine how deeply the swab is being inserted in a nostril. In the embodiment shown in FIG. 1, only one swab 115 is provided since many nosebleeds only involve one nostril. Clip 130 is applied in an orientation that aligns swab 115 with the bleeding nostril.

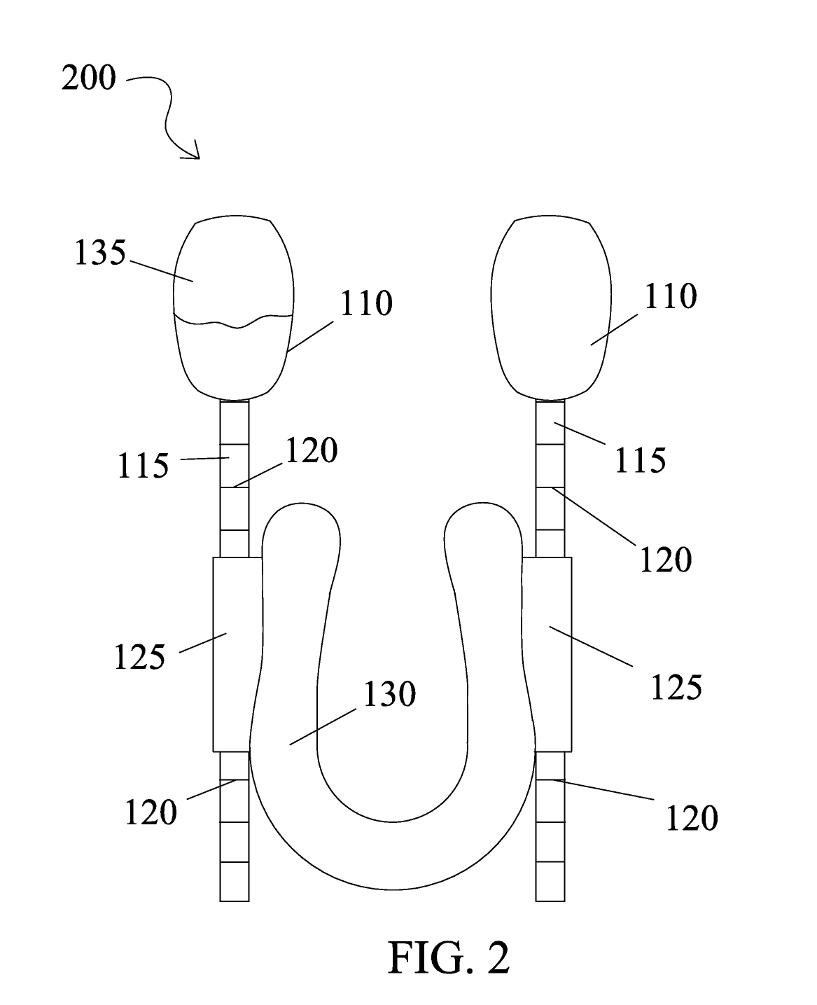

Referring now to FIG. 2, a nose bleed treatment system 200 has two swabs 115 that fit within swab holder 125 that are disposed on both sides of clip 130. Medication 135 may be applied to one side or both depending on the bleed encountered. In operation, this embodiment is essentially the same as discussed above with respect to single swab embodiment.

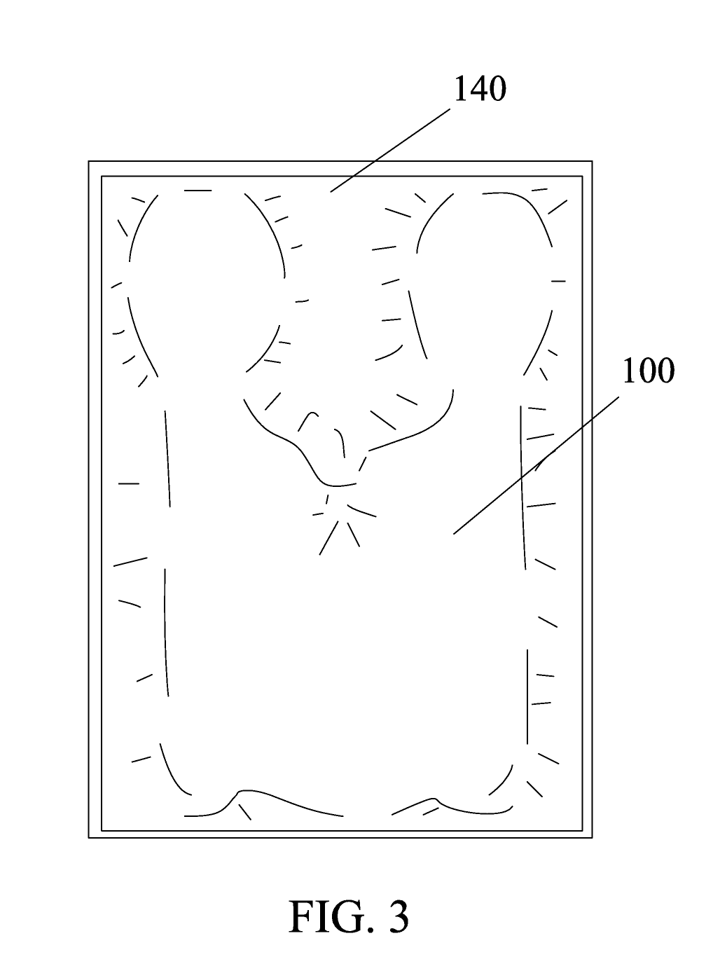

Now referring to FIG. 3, a hermetically sealed package 140 keeps nose bleed treatment system 100 germ free and keeps the medication effective and ready for use by tearing open the package. In this embodiment, the medication is pre-applied to the swab. Package 140 is sealed using vacuum sealing methods as is known in the art.

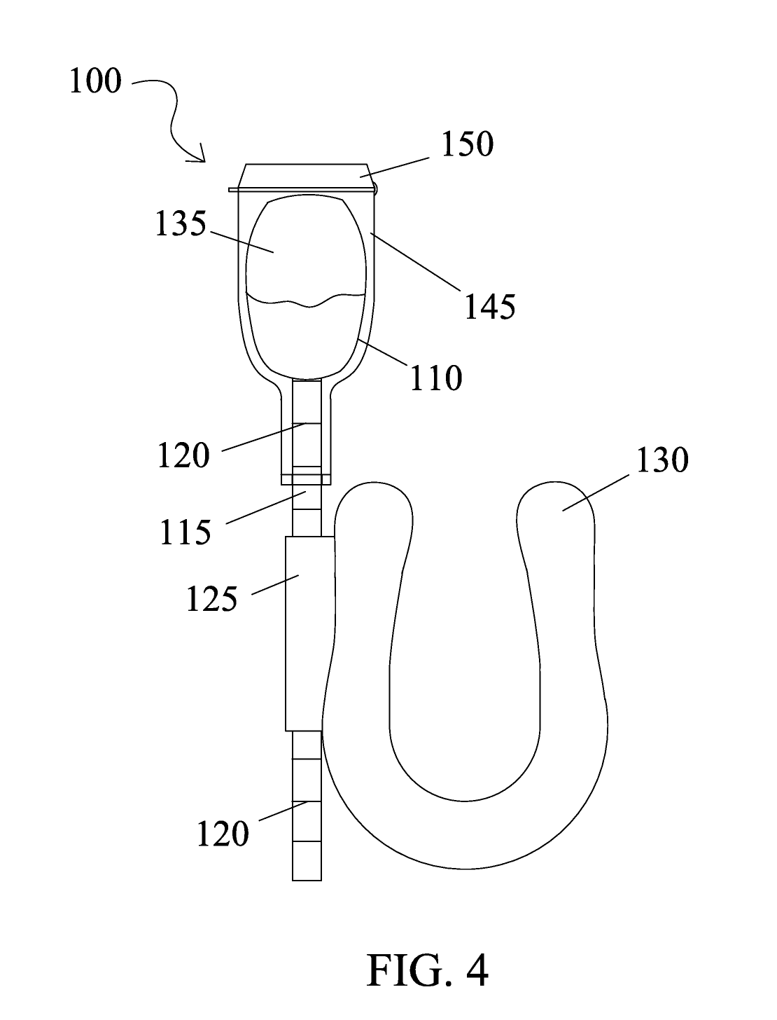

Referring now to FIG. 4, the nose bleed treatment system 100 is shown with a hermetically sealed swab protector 145 that keeps medication 135 efficacious and ready for use. A hinged cap 150 is provided that opens when needed and then slips down and off the stick portion of swab 115. Of course, swab protector 145 may be used with the two swab embodiment shown in FIG. 2.

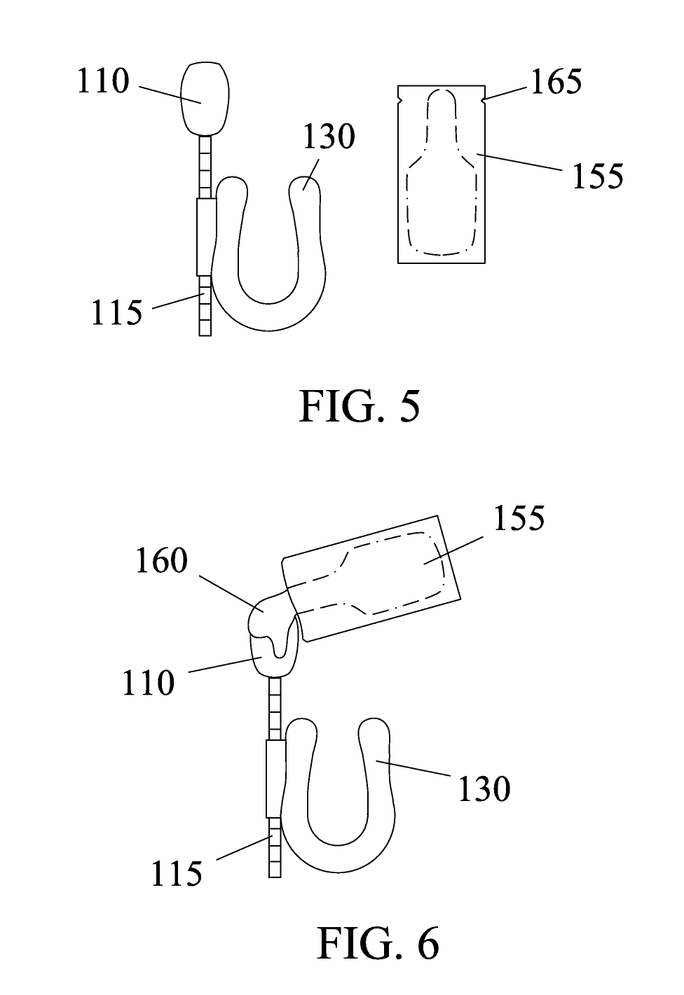

Referring to FIGS. 5 and 6, a medication packet 155 is provided to be used with clip 130, and swab 115. A tear strip 165 is provided to allow a user to tear open medication packet 155 and apply medication 160 to swab tip 110 when ready to use.

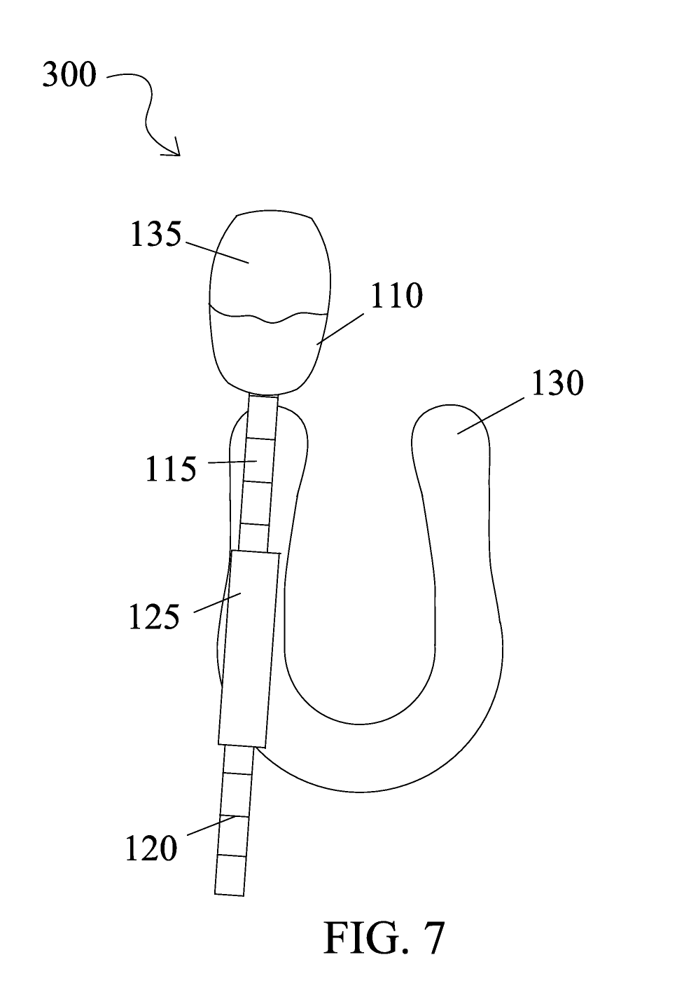

Referring now to FIG. 7, a nose bleed treatment system 300 is shown placed in an alternative location to facilitate insertion of swab 115 in the affected nostril. Of course, it should be understood that other placements may be used to allow for comfortable placement of swab in the user's nostrils without departing from the spirit of the present invention.

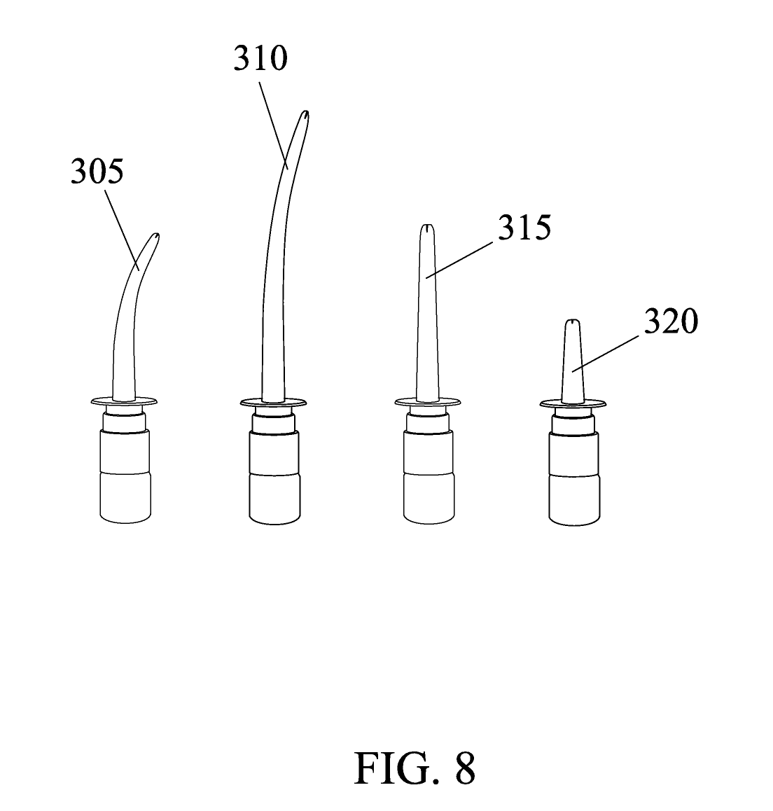

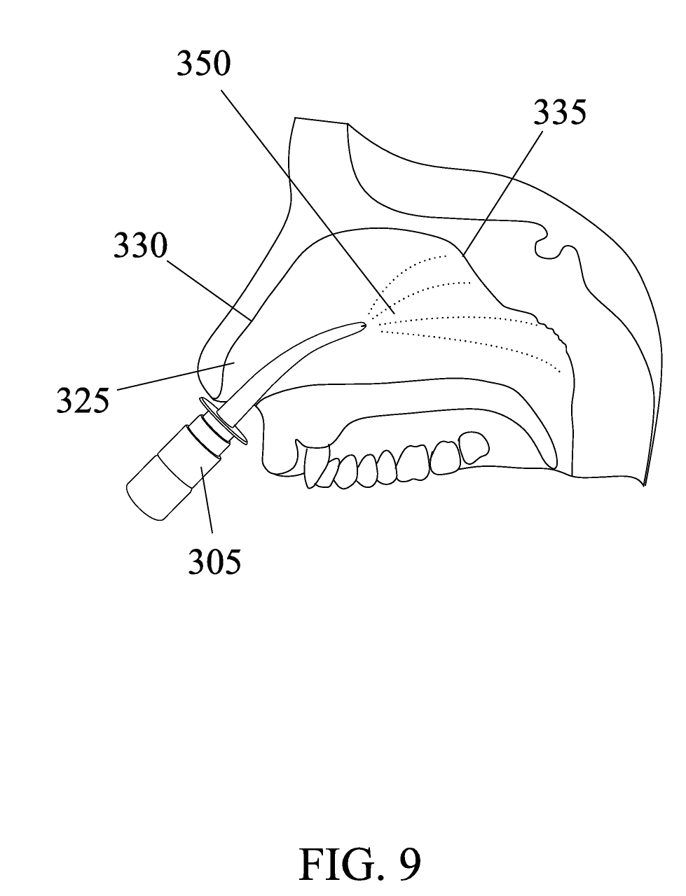

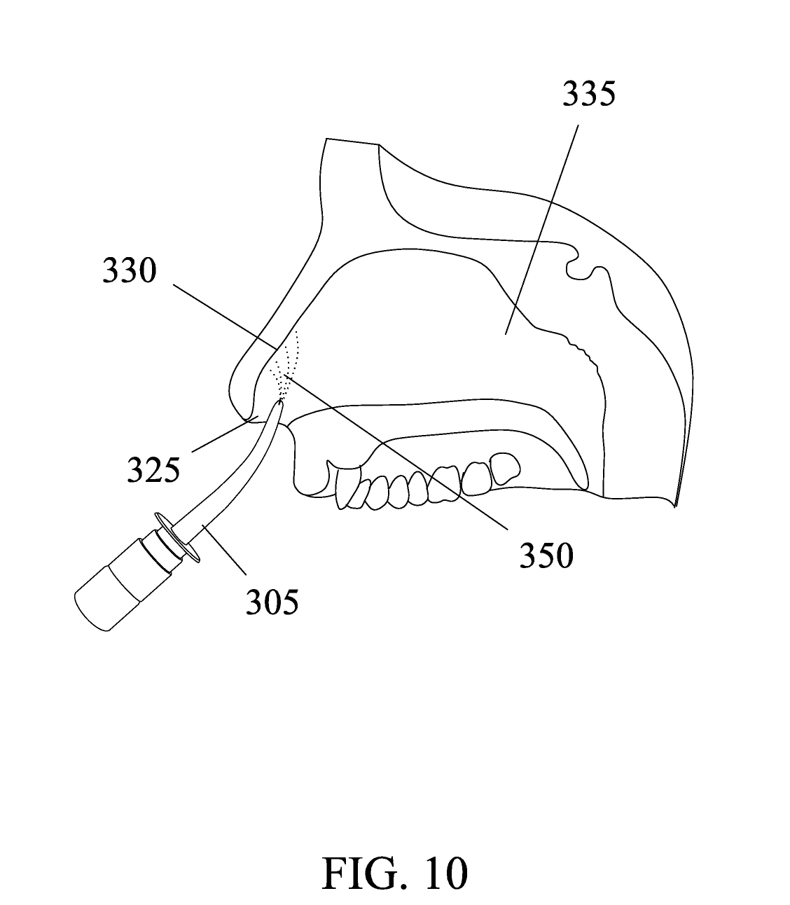

Now referring to FIG. 8-11, nose spray bottles are shown having different shaped nozzles to accommodate different shaped nasal cavities. A nasal spray 305 is shown having a curved portion to allow a user to insert in a nasal cavity 325 to deliver a spray dose 350 of a vasoconstricting medication. FIG. 9 illustrates delivery of spray dose 305 to a posterior portion 335 of nasal cavity 325 to help stop a posterior nosebleed. FIG. 10 illustrates delivery of spray dose 350 to an anterior portion 330 of nasal cavity 325 for treatment of the more common anterior nosebleed. The user rotates spray bottle 305 to deliver spray dose 350 to the required area depending on the nosebleed. Again as discussed above, posterior nosebleeds, though less common than anterior, are generally more serious and more difficult to treat since they occur deep within nasal cavity 325.

Spray bottle 310 has a very long delivery portion to aid in delivering the medication to the required area. Of course other shapes may be used as shown by spray bottle 315 and spray bottle 320 to match the physiology of the user such as children, person with a deviated septum, etc. as long as the delivery portion is able to reach the affected area of the nasal cavity.

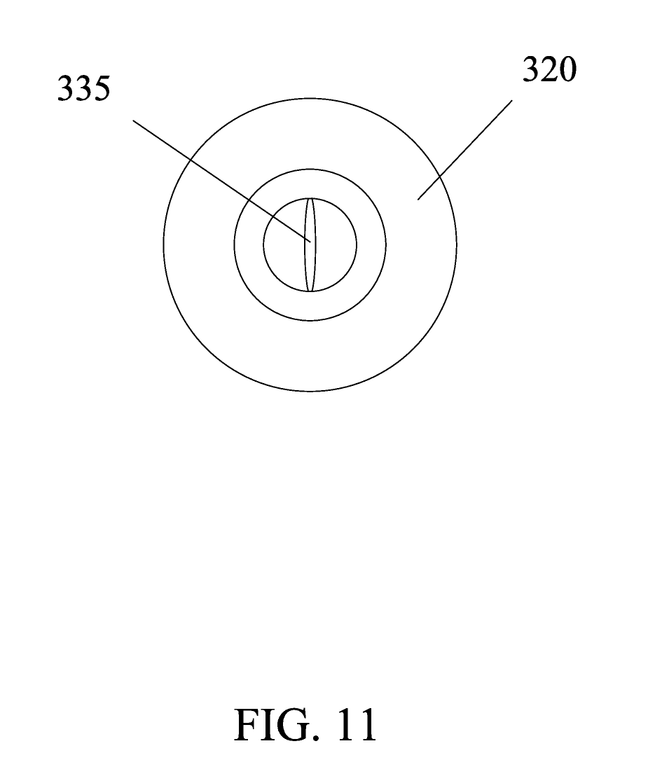

Additionally, as shown in FIG. 11, a tip 340 is an elongated slit to aid in directionally applying the medication to the desired location.

This greatly enhances the ability to treat problematic nosebleeds. With tip 340, spray dose 350 is sprayed in a flattened pattern rather than in a cone as would be provided by a circular hole. This allows spray dose 350 to be applied more precisely which is more comfortable and effective for the user. Again as discussed above, spray dose 350 contains a medically effective agent which may contain vitamin K as well as a vasoconstricting agent epinephrine with pytonadione (vitamin K1), menaquinone (vitamin K2), menadion (vitamin K3) fibrogen or other suitable medications.

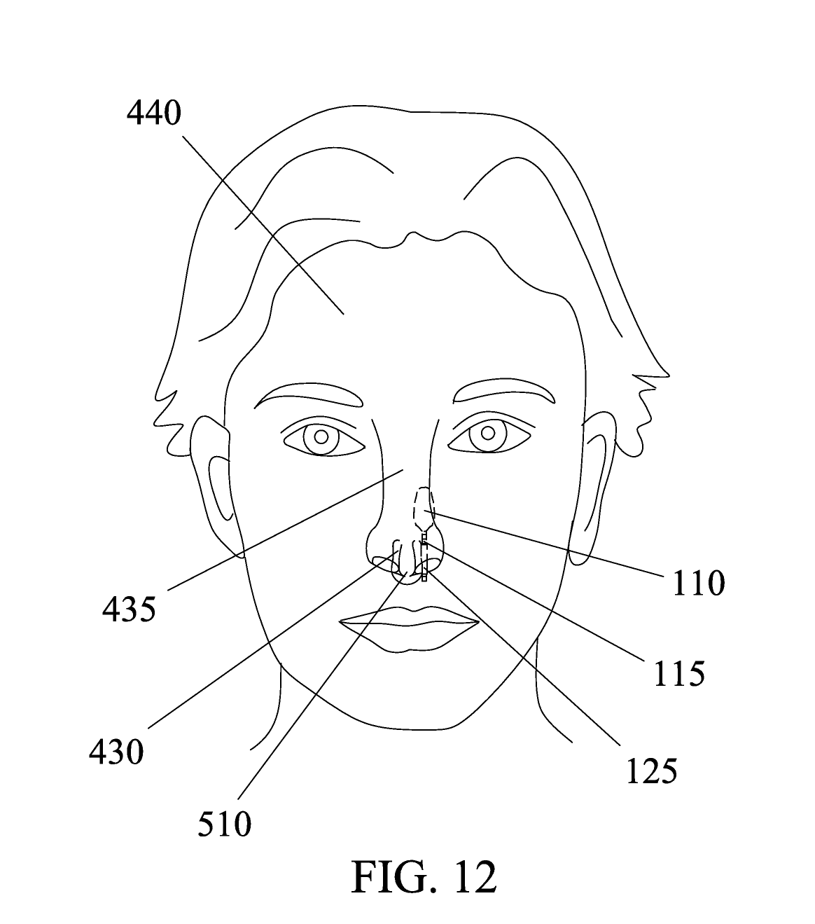

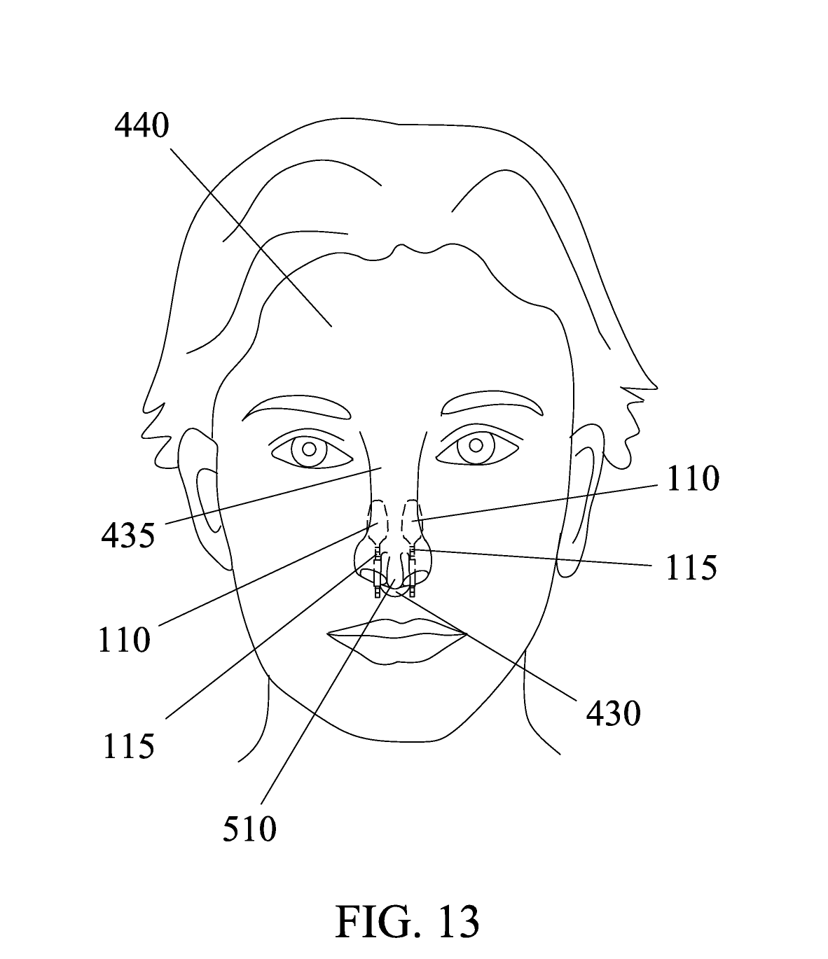

Now referring to FIGS. 12 and 13, a patient 440 is positioned in an orientation that allows the user to most easily reach the nostrils and apply the agent and place the clip on the nostrils. It is often desirable to have the patient bend their head backwards to more easily allow treatment. The user selects a tip most suitable for the patient to enable the user to apply the agent close to the source of the bleeding. This can depend on the physiology of the patient. Once the agent has been applied, a nose clip 430 is stretched open and gently applied to the patient's septum 510 inside the patient's nose 435. Clip 430 holds swab 115 in the desired location with swab tip 110 pressed against the site of the bleeding. In use, very little of swab 115 is visible and once swab 115 is placed in position, it may be trimmed off so that it does not protrude past the nasal opening. Swab 115 may be pre-stressed to provide for tool free trimming by having break rings manufactured therein. In use, once the desired swab length is selected, the swab is broken at a corresponding break line to trim it to the desired length. FIG. 13 is an embodiment of nose clip 430 having two swabs 115 that allow the user to place swabs 115 in each nostril and corresponding nasal cavity 325.

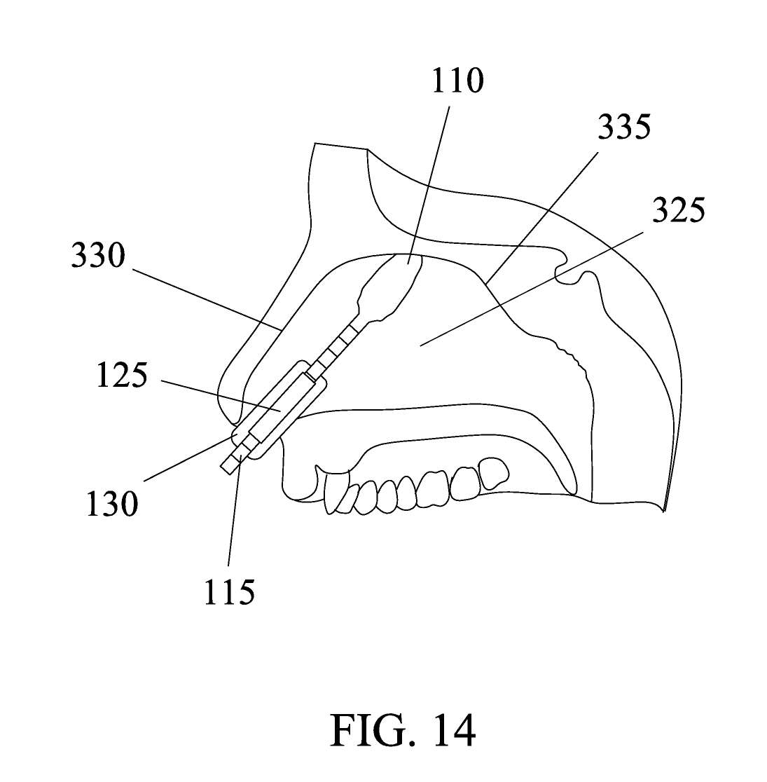

As can be seen in FIG. 14, the location of swab 115 may be placed against a bleeding site in both an anterior portion 330 (more common) and posterior portion 335 (less common but more serious). Clip 130 may be adjusted to allow the user to place swab tip 110 against the bleed site wherever it is located. Proper placement is obtained by moving clip 130 in combination with moving swab 115 in swab adjustment holder 125 so that swab tip 110 may be placed against a bleed site located anywhere within the patient's nasal cavity 325 including the more dangerous and difficult to reach posterior portion 335. The medicated agent may be applied using spray bottle 305 or applied to swab tip 110 or both as desired. In this way, swab tip 110 is pressed against the bleed site and in combination with the medication and pressure; the blood flow is reduced and then stopped.

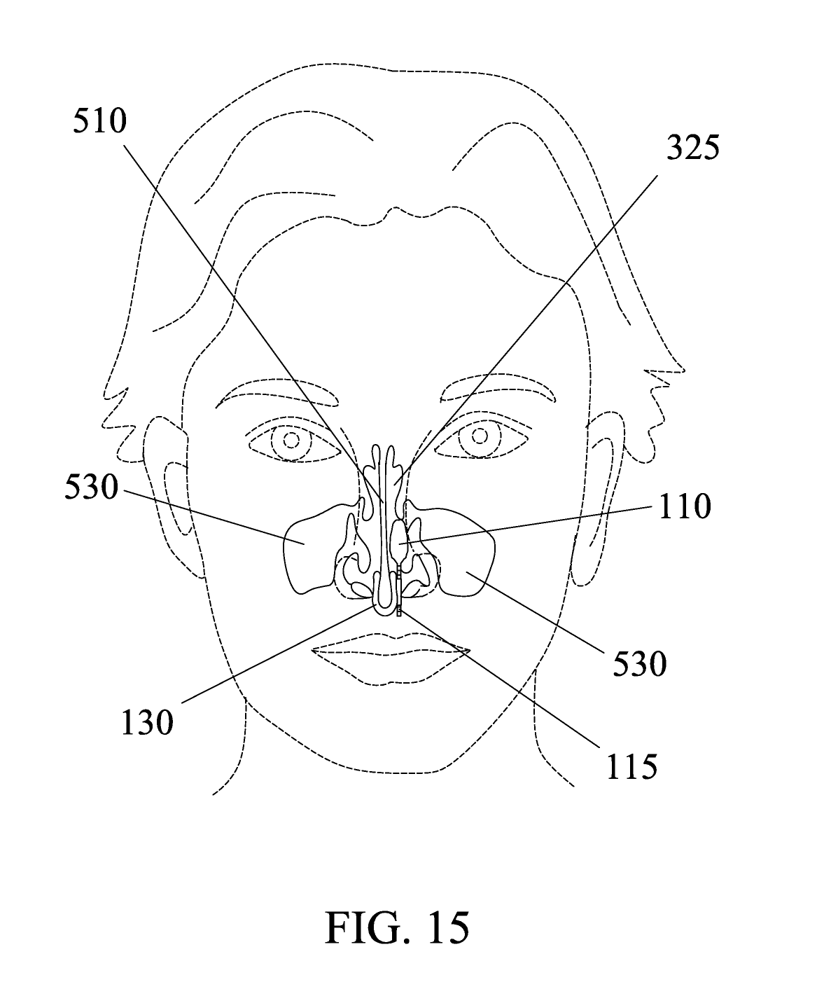

FIG. 15 further illustrates positioning of swab tip 110 and clip 130 within nasal cavity 325 and sinuses 530 in use with a patient.

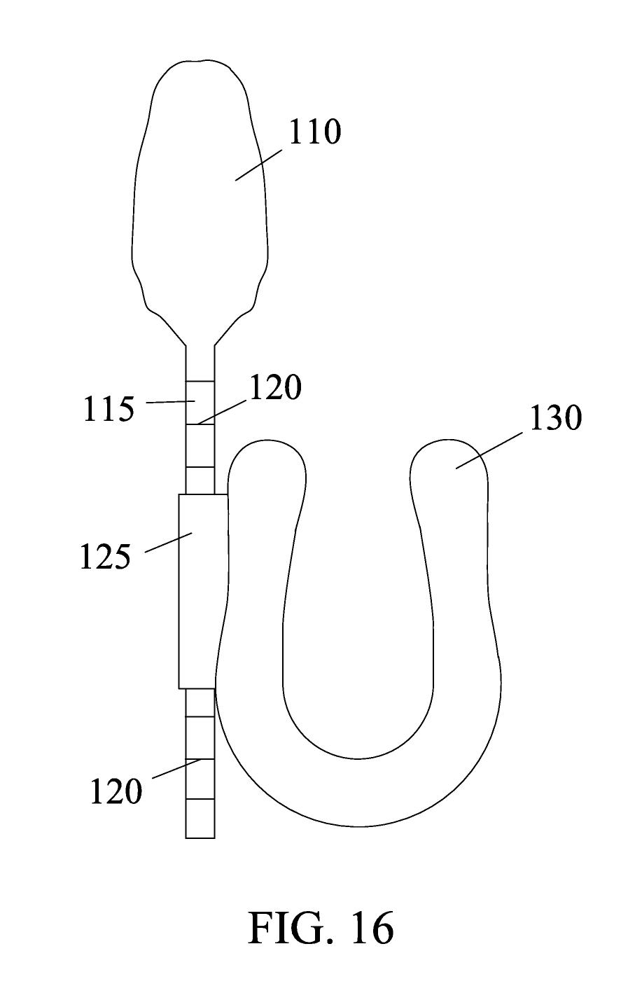

FIG. 16 illustrates the use of a bulky swab tip 110 to allow more medication, pressure and blood absorption than is possible using a smaller swab tip.

Although the instant invention has been described in relation to particular embodiments thereof, many other variations and modifications and other uses will become apparent to those skilled in the art.

1. A nose bleed treatment system for use in treating a patient having a nose bleed;
comprising a nose clip,
said nose clip having at least one swab adjustment holder disposed therein;
a swab adjustably and longitudinally disposed within each of said at least one swab adjustment holder;
said swab having a swab tip and a handle portion;
said handle portion adapted to move longitudinally within a nasal cavity of a patient;
said swab handle having a plurality of measurement indication lines disposed thereon;
said plurality of measurement indication lines adapted to indicate the insertion depth of said swab tip; and
said swab tip adapted to dispense a medically effective agent for application to a bleed site.

2. The nose bleed treatment system for use in treating a patient having a nose bleed according to claim 1 wherein said medically effective agent contains a clot enhancing agent.

3. The nose bleed treatment system for use in treating a patient having a nose bleed according to claim 1 whereby said medically effective agent is delivered to said bleed site using a spray bottle having an elongated tip which fits within the nostril of the patient.

4. (canceled)

5. The nose bleed treatment system for use in treating a patient having a nose bleed according to claim 2 wherein said clot enhancing agent is vitamin K.

6. The nose bleed treatment system for use in treating a patient having a nose bleed according to claim 1 wherein said medically effective agent contains a vasoconstricting agent.

7. The nose bleed treatment system for use in treating a patient having a nose bleed according to claim 6 wherein said vasoconstricting agent is an herbally sourced ephedra compound.

8. The nose bleed treatment system for use in treating a patient having a nose bleed according to claim 5 wherein said herbally sourced ephedra compound is a vasoconstricting agent containing phenylephrine/ephedrine.

9. The nose bleed treatment system for use in treating a patient having a nose bleed according to claim 3 wherein said vitamin K is from an herbal source.

10. The nose bleed treatment system for use in treating a patient having a nose bleed according to claim 2 wherein said clot enhancing agent is a mammalian sourced serum containing fibrinogen.

11. The nose bleed treatment system for use in treating a patient having a nose bleed according to claim 1 wherein said medically effective agent contains a clot enhancing agent and a vasoconstricting agent.

12. The nose bleed treatment system for use in treating a patient having a nose bleed according to claim 1 wherein said medically effective agent is selected from the group consisting of vitamin K, epinephrine with pytonadione (vitamin K2), menaquinone (vitamin K2), menadione (vitamin K3) and fibrogen.

13. (canceled)

14. (canceled)

15. (canceled)

16. (canceled)

17. (canceled)

18. (canceled)

19. (canceled)

20. (canceled)
